(19) United States
(12) Patent Application Publication
(10) Pub. No.: US 2012/0035636 A1
(43) Pub. Date: Feb. 9, 2012

(54) DEVICE FOR CUTTING THE CORNEA OF AN EYE

(76) Inventor: Albert Daxer, Linz (AT)

(21) Appl. No.: 13/138,458

(22) PCT Filed: Feb. 19, 2010
(86) PCT No.: PCT/EP2010/052123

§ 371 Date: Aug. 19, 2011

(30) Foreign Application Priority Data
Feb. 20, 2009 (AT) A 285/2009
Oct. 21, 2009 (AT) A 1661/2009
Nov. 6, 2009 (AT) A 1766/2009
Nov. 17, 2009 (AT) A 252/2010

(51) Int. Cl. A61F 9/013

(52) U.S. Cl. 606/166

(57) ABSTRACT

The invention relates to a device for cutting the cornea of an eye in order to correct the refractive power thereof, with a ring body that can be suctioned onto the eye, with an applanator (7) for deforming the cornea, and with a blade (4) which is guided in a guide plane perpendicular to the axis of the ring body, is mounted in front of the applanator (7) and is used to cut a pocket in the corneal tissue. So as to avoid adversely affecting the cutting depth and therefore the cutting accuracy upon penetration of the holder into the corneal pocket, provision is made that the blade (4) is secured on a blade receptacle (50) of a holder or fixture in such a way that the top side (51) of the blade receptacle (50) does not protrude above the top side (44) of the blade (4), at least within the depth of penetration of the blade receptacle (50) into the cornea.

### FIELD OF THE INVENTION

The present invention relates to a device for cutting the cornea of an eye in order to correct the refractive power thereof, with a ring body that can be suctioned onto the eye, with an applanator for deforming the cornea, and with a blade which is guided in a guide plane perpendicular to the axis of the ring body, is mounted in front of the applanator and is used to cut a pocket in the corneal tissue.

The applanator can be designed as a stamp for deforming the cornea within the ring body.

The ring body and the applanator can both be arranged in a frame.

The blade can be secured in a holder, which is guided on the frame in a guide plane perpendicular to the axis of the ring body, wherein the blade can pass through a peripheral recess in the frame and is mounted in front of the applanator. Moreover, in order to cut into the corneal tissue a pocket having merely a tunnel-like access, the blade is on the one hand movable radially with respect to the ring body via the holder and on the other hand pivotable about an axis perpendicular to the guide plane at each blade position within an area of a cutting plane parallel to the guide plane, wherein the blade passes through the peripheral recess with clearance, and the holder supports a vibrator for an oscillating movement of the blade in the cutting plane.

### PRIOR ART

In order to be able to correct the refractive power of the cornea of an eye, it is known (US 2001/0004702 A1) to treat the interior of the corneal tissue via an access route having a width of, for example, ca. 4 mm. A pocket is created in the interior of the cornea, which pocket can be widened to form a foldable corneal flap and thus permits surgical access to the corneal stroma (deep corneal tissue) for the purpose of correcting the refractive power.

To be able to perform such correction of the refractive power, US 2001/0004702 A1 proposes securing a frame on the eye of a patient, on which frame a holder for a movable blade is secured. The blade passes through the frame via a peripheral recess and is guided in a guide plane via a holder. This guide plane lies parallel to the cutting plane of the blade, a pocket being produced in the cornea in said cutting plane. The blade in its cutting plane is additionally held down by a spring so as to rest on the frame, since a slipping of the blade from where it is applied to the cornea for producing the tunnel-like access must be avoided. It is in fact quite difficult to penetrate the elastic and extremely tough outer layer of the cornea. In addition, the blade must not bend during cutting, since in that case it is not possible to guarantee an exact cutting plane and to avoid perforation of the outer or inner layer of the cornea, which precludes successful correction of the refractive power. While this may be counteracted using blades of great hardness (for example made of diamond material), such blades are particularly sensitive to shear stresses, which excludes the use of such blades in the device according to US 2001/0004702 A1, given the fact that the blade rests on the frame. Moreover, in addition to the risk of their breaking, the sharpness of such blades would also be greatly impaired.

It is also known from the prior art (WO 2004/096106 A1) to design a somewhat narrow and pivotable blade with a vibrator and to guide it in such a way that the blade edge cannot be impaired even in the case of hard materials. A device for cutting the cornea of an eye in order to correct the refractive power thereof was thus already created with which, starting from the site where the blade was applied to the cornea, a pocket lying in the cutting plane of the blade can be cut with accurate positioning through a tunnel-like access, without any reduction in the stability of the cornea having to be considered. Moreover, the device permits a wide range of corrections of the refractive power. Compared to other methods of correcting the refractive power, where either the surface of the corneal tissue is ablated (DE 34 33 581 A1) or the cornea is broadly incised to produce a flap that may be folded back (LASIK method), treating the interior of the cornea via a tunnel-like access has the advantage that virtually no post-operative pain occurs and/or the stability of the cornea is scarcely impaired, since the top layer, which is important for the stability of the cornea and which also includes the Bowman membrane for example, remains uninjured for the most part. Therefore, treating the cornea via a tunnel-like access offers many advantages over the other methods of treatment.

In addition, the device permits a wide range of refractive power corrections.

Moreover, U.S. Pat. No. 6,599,305 B1 discloses a device in which a narrow access to a corneal pocket is possible only in very narrow corneal pockets and, consequently, a relatively wide access, with impairment of the biomechanics, is necessary in usable optical zones. Moreover, in clinical use, the principle and the design of the cutting device with blade and blade holder mean that perforations of the cornea must be expected as serious complications.

US 2001/0004702 A1, WO 2004/096106 A1 and U.S. Pat. No. 6,599,305 B1 do not solve the problem of how to secure the blade on the holder in such a way that, when the holder penetrates into the corneal pocket, the cutting depth and therefore the cutting precision are not impaired, and, at the same time, of how multiple sterilization can be achieved.

### DISCLOSURE OF THE INVENTION

The invention solves the stated problem by virtue of the fact that the blade (in the sense of a blade that has to be secured on a receptacle) is secured on a blade receptacle of a holder or fixture in such a way that the top side of the blade receptacle does not protrude above the top side of the blade, at least within the depth of penetration of the blade receptacle into the cornea.

The top side of the blade receptacle or of the blade is that surface of the blade receptacle or of the blade that is directed towards the contact face of the applanator. Correspondingly, the underside of the blade receptacle or of the blade is that surface of the blade receptacle or of the blade that is directed away from the contact face of the applanator.

The blade receptacle ensures that the blade does not have to be mounted directly on a movable holder, such as that with reference sign 3 in FIG. 1, or does not have to be mounted directly on a fixture, such as that with reference sign 83 in FIGS. 18 to 21, and instead the blade receptacle forms an intermediate piece for indirect securing of the blade on the holder or fixture. The blade is secured at a first location on the blade receptacle and mounted on the holder or fixture at a second location different from the first location. The blade receptacle thus functions as an intermediate piece between blade and holder/fixture.

In one design variant, provision can be made that the blade and the blade receptacle each have a preferably flat surface on the top side, and the blade has a flat surface on the underside, in which case the cutting edge of the blade is equidistant from the two flat surfaces of the blade and, during the cutting procedure, the distance between the highest point of the flat surface on the top side of the blade receptacle and the contact face of the applanator, measured perpendicular to the cutting plane, is not less than the distance between the flat surface on the top side of the blade and the contact face of the applanator, measured perpendicular to the cutting plane.

In order to ensure a good connection between blade and blade receptacle, provision can be made that the blade is provided with a shaft, which shaft is adhesively bonded into the blade receptacle. Provision can additionally be made that the shaft is roughened on the surface within a partial area, on at least one side, and the roughening is adhesively bonded to the blade receptacle.

However, it is also possible that the blade has no shaft in the real sense, in other words the blade has a blade edge along its entire longitudinal extent (see FIG. 22). In this case too, provision can be made that the blade is roughened on the surface within a partial area, on at least one side, and the roughening is adhesively bonded to the blade receptacle.

Roughening is to be understood as an increase of the average surface roughness, in relation to the rest of the blade surface (or, in blades with a shaft, in relation to the cutting area), by at least a factor of 1.1. Still better connections can be achieved with roughening by a factor of 2 to 100 or even higher. The roughening can have any desired macroscopic or microscopic geometry. Microscopically, it can be in the form of furrows, nipples and other elevations and depressions. The height between the lowest and next highest location (peak-to-valley heights) of the roughening should preferably be over 40 nm. Ideally, the peak-to-valley heights of the roughening are between 100 nm and 600 nm, in particular 400 nm. In particular embodiments, it can also be in the micrometre range, but preferably less than 10 μm (micrometre). Ideally, the roughening is produced by means of laser radiation. However, it can also be produced chemically by etching or mechanically or in another way.

Provision can additionally be made that the blade receptacle is likewise roughened in an area corresponding to the roughened area of the blade and/or of the shaft. The roughening and the way in which the latter is produced correspond to those of the blade.

Suitable adhesives are all those that can ensure sufficient strength and hardness even under conditions of steam sterilization. For example, epoxy adhesive or epoxy resin adhesive can be used. Such an epoxy adhesive should preferably be provided as a two-component adhesive consisting of resin and hardener, for example with 100 parts by weight of resin and 10 to 20 parts by weight, most preferably 13 parts by weight, of hardener. Aluminium oxide can be used as filler. The adhesive should be such that the hardening at room temperature does not take more than 24 hours, or at 250° C. takes ideally less than 3 hours. The viscosity before hardening can be relatively high (pasty) but can also lie in the range of 10000-30000 cps, preferably about 20000 cps, which, with 1 cps=10⁻³ kg/ms, corresponds to a viscosity of ideally about 20 kg/ms or a range of 10-30 kg/ms. The thermal expansion of the finished adhesive compound should be as low as possible and should not exceed 10×10⁻⁵/° C. In a particular embodiment, it is 6.4×10⁻⁵/° C. Ideally, it is not more than 5×10⁻⁵/° C., approximately between 2 and 4×10⁻⁵/° C. The adhesive compound should be able to be sterilized more than once and should have a deformation temperature of at least 150° C. (ideally at least 200° C.) The adhesive compound should have a density of less than 2.5 g/cm³ (between 1 and 2 g/cm³), e.g. 1.9 g/cm³. The volume shrinkage of the adhesive in the hardening process is less than 1% (ideally 0.3% or less, e.g. 0.2%). The hardness should be between D70 and D90, ideally about D80 or D85. The weight loss over a period of 500 hours at sterilization temperatures in excess of 134° C. should be below 1% (ideally 0.5% or less), e.g. 0.5% over 1000 hours at 200° C.

By way of the roughened part of the blade shaft or of the blade, it is possible to connect the blade to the blade receptacle with a suitable adhesive compound in such a way that the blade receptacle, on the side directed towards the applanator or towards the contact face (applanation face) thereof, has a depression corresponding to the shaft geometry (see FIG. 14) or to the blade geometry (see FIG. 22). The extent of this depression should cover at least an area of 1 mm². In any case, the surface area of the depression is at least as great as the surface area of the shaft of the blade. The depth of the depression in the blade receptacle should be configured such that, when an adhesive connection is made, the surface of the blade receptacle directed towards the contact face (applanation face) does not protrude further, in the direction of the contact face (applanation face), than the corresponding surface of the blade directed towards the contact face.

If the cutting edge is not located on the surface, a further improvement of the cutting function is possible by virtue of the fact that the top side of the blade receptacle does not protrude above the cutting edge.

Since the applanator prevents a migration of the tissue in front of the blade during cutting, this means that, when the blade penetrates into the cornea, the tissue has to migrate “rearwards”, that is to say in the direction of the anterior chamber of the eye, or the blade retreats relative to the surface and there is the danger of an unequal cutting depth and/or the danger of perforation of the cornea. If the top side of the blade receptacle does not protrude above the top side of the blade or the cutting edge of the latter, the blade, upon deep penetration into the cornea, does not drop away from the contact face (applanation face) and the incision depth remains constant at the front of the blade. This permits an exactly guided incision and a precisely defined corneal pocket. Unequal depths on the opposite sides of a pocket can lead to errors in the refractive power correction and to complications such as extrusion of annular implants through the corneal surface.

By virtue of the fact that the top side of the blade receptacle does not protrude above the top side of the blade or the cutting edge of the latter, it is also possible that, despite penetration of the blade receptacle into the cornea, an exact cut is achieved. This in turn means that shorter blades can be used, which increases the stability of the blades in terms of cutting accuracy. In fact, if the blade receptacle cannot penetrate into the cornea, only comparatively long blades can be used to produce the pocket. The longer a blade, the more “bendable” it is, and a constant cutting depth is maintained with less precision. This is of particular importance when making an incision in the cornea, since the thickness of the cornea is only ca. 500 μm (0.5 mm) and any deviation from the ideal cutting depth can have fatal consequences for the eyesight. It must also be considered that the blade is applied to an oblique and tough surface (corneal surface outside the applanation area) for penetrating into the cornea, and this in itself leads to a considerable deviation from the ideal direction of penetration, because of the resulting bending moment on the blade. For this reason, metal blades, even when diamond-coated, which is after all only a surface measure, are somewhat problematic because of their ductility. Even diamond blades, despite their great hardness, can have a certain bendability if they are long, thin and narrow. The invention thus increases the stability of the blades with the objective of increasing the cutting precision.

By designing the blade as a long blade (i.e. as an elongate and somewhat narrow blade) with a blade edge along at least one longitudinal side, a drawn incision can be made. Blade edge designates the areas that are bevelled in relation to the flat top side and underside of the blade and that form the cutting edge at their line of intersection. The blade width should not exceed 4 mm. Blade widths of 2 to 2.5 mm are ideal. In particular embodiments, for example for small pocket widths (less than 6 mm) and a depth of penetration of the pocket of just over the halfway point of the cornea, the blade width can also be up to 6 mm and over.

The thickness of the blade should be between 100 and 250 μm, ideally 150 μm to 200 μm. The length of the shaft should be 1 to 3 mm, ideally 2 mm. The shaft is ideally the same width as or narrower than the blade in the cutting area. It can have the same thickness as or be thinner than the blade in the cutting area.

If the underside of the blade receptacle, that is to say the side of the blade receptacle directed away from the applanator, slopes away from the shaft and from the blade, this has the advantage that, during the movement of the blade receptacle in the pocket, the tissue displaced by the underside of the blade receptacle does not move abruptly rearwards (in the direction of the anterior chamber of the eye), and this likewise considerably improves the cutting precision and safety for the eye. It is best if no step is present on the underside of the blade at the transition between the blade receptacle and the blade, and the thickness of the blade receptacle thus constantly increases, from the zero thickness at this point, as the distance from the blade tip increases.

Alternatively or in addition, the blade receptacle can also slope from the lateral edges (the edges parallel to the longitudinal axis of the blade) towards the centre of the blade receptacle. In other words, the thickness of the blade receptacle can increase on the underside from the lateral edges of the blade receptacle towards the centre. This has the advantage that the tissue is not moved abruptly upon a lateral movement of the blade. Such a design is present, for example, when the shape of the blade edges and cutting edges is continued beyond the blade on the blade receptacle.

If, alternatively or in addition, the top side of the blade receptacle, at least as far as the blade receptacle penetrates into the cornea, is flat and preferably extends parallel to the cutting plane, this has the advantage of having no effect on the tissue in front of the blade (between blade and corneal surface).

It is also advantageous if the blade is made of a particularly hard material, for example diamond, ruby, semiconductor materials or ceramic. However, in addition to being made of non-metallic materials, the blade can generally also be made of metallic materials. Comparatively unfavourable cutting conditions arise with ductile or metallic blades, even if they have diamond-coated surfaces or cutting edges. However, metallic blades can be very easily connected to a blade receptacle by means of adhesives. By contrast, it is known that diamond blades can be adhesively bonded to blade receptacles only with great difficulty and also only for a limited period of use (sterilization cycles). Normally, in order to achieve a good adhesive connection, the shaft of the diamond blade is enclosed on all sides by the blade receptacle, and this enclosure is often strengthened by means of an adhesive connection. However, enclosing the blade or shaft in the blade receptacle also from the top side leads to the abovementioned considerable inaccuracy of cutting, with the risk of corneal perforation.

The blade receptacle can be made of any desired material, preferably of metal (steel), but also of other materials, such as ceramic (ZrO), ruby or hard plastic. In the area of the blade shaft, or also further away from the blade, the blade receptacle can be narrowed in relation to the cutting blade width. However, alternatively or in addition, the blade receptacle can be laterally shaped into a blade edge or ground.

The blade receptacle can be an integral part of the blade holder guided on a frame, i.e. can be designed in one piece with the holder.

If the preferably non-metallic blade forms a pointed tip, this facilitates the penetration of the blade from the place where it is applied to the cornea. If the blade additionally has a blade edge tapering towards the pointed tip, it is possible, by moving the blade radially with respect to the ring body, to produce a particularly narrow tunnel-like access into the cornea, which is useful for the stability of the cornea.

By designing the blade as a long blade with at least one blade edge, but preferably two blade edges extending along the longitudinal sides, with corresponding cutting edges which taper to a pointed tip at the end remote from the blade receptacle, provision can be made that the cutting edges are equidistant from the upper side of the blade directed towards the applanation face and the lower side of the blade directed away from the applanation face. The cutting edges can preferably extend parallel to each other along both longitudinal sides of the blade and parallel to the longitudinal axis of the blade and taper towards the pointed tip at an angle alpha of less than 90°, ideally ca. 70° (between 60 and 80°), at the end directed away from the blade receptacle. The blade edge (or more precisely the face of the blade edge) is bevelled with respect to the surface by an angle beta of in each case 10° to 25° (ideally 15° to 20°). From the shaft of the blade to the tip of the blade (the end directed away from the blade receptacle), the cutting edge, independently of the position of the cutting edge, should have a length of at least 3 mm and at most 8 mm, preferably 5-7 mm.

In a particular embodiment, the cutting edge can preferably be continued with an identical profile onto the blade receptacle. The continuation of the cutting edge onto the blade receptacle can be up to 7 mm, ideally 5 mm, such that a blade can effectively produce a cut of up to 12 mm (ideally between 5 mm and 12 mm). In particular, the blade receptacle can have another blade, i.e. a second blade, in which the cutting edge of the actual blade continues. This second blade can be mounted, for example, on the top side of the rest of the blade receptacle.

By providing a two-part receptacle for the applanator, wherein the two parts together form a peripheral groove for guiding a holder for the blade, the two parallel surfaces of the peripheral groove, between which the blade is moved precisely and practically free of clearance, can be made parallel with a very high degree of precision.

In a preferred embodiment, the blade and the blade receptacle pass with clearance through the peripheral recess of the frame, which ensures that the blade, when it is moved, particularly when it is inserted into its cutting position, does not rest on the frame, such that damage to the blade or to the blade edge can be ruled out. It is therefore possible to use comparatively hard materials for the blade, since the brittleness of these mostly very hard materials can in fact be ignored. In contrast to US 2001/0004702 A1, therefore, a fracture or damage to the blade edge does not have to be taken into consideration, since the blade is guided with its sensitive parts free from contact, as a result of which, according to the invention, blades with high cutting performance and small dimensions can be used. When penetrating the outer layer of the cornea in order to produce a tunnel-like access, it is thus also possible to assume that the tunnel-like access is extremely precisely contiguous to the site where the blade has previously been applied to the cornea.

The penetration of the blade is made easier by the fact that the holder, preferably in the area of the blade receptacle, supports a vibrator for a vibration of the blade in the cutting plane. This in particular serves to overcome the elasticity of the outer layer of the cornea without risking increased indentation of the corneal surface. The vibrator also ensures that minimal force is exerted during the cutting procedure, such that it is possible to exclude any movement of tissue that is attributable to the motion of the blade resulting from the elasticity and toughness of the tissue and that arises even when using extremely sharp blades. Thus, an especially drawn incision for high cutting precision can be ensured.

If the receptacle is designed as an exchangeable receptacle in which applanators with differently curved contact faces for deforming the cornea can be received in a manner limited by a stop, wherein the contact faces in successive incisions define pockets for delimiting a lens-shaped portion of tissue, it is possible, starting from a tunnel-like access, to cut out a volume of tissue without having to produce a corneal flap. The lens-shaped portion of tissue that is cut out can then be pulled out via the tunnel-like access, as a result of which it becomes possible, according to the invention, to produce a defined cavity in the cornea, without significantly impairing the stability of the cornea in comparison to the other methods. This makes it possible to create a tissue lens in the cornea through which a refractive error can be corrected.

If the peripheral edges of the contact faces of the applanators inserted in succession into the receptacle have different perpendicular distances from the cutting plane of the blade, the cutting out of a lens-shaped portion of tissue is thus made easier. Although this difference in the perpendicular distances requires two tunnel-like accesses to be produced in the corneal tissue, this nevertheless has the advantage that the two pockets are prominent in their common sectional line, which excludes the possibility of only partial cutting out of the volume of tissue.

If the applanator is made of transparent material, this allows the surgeon to easily observe the contact face and/or the cutting process. If the applanator is additionally designed as a magnifying lens, the focal point of which lies in the area of the contact face for deforming the cornea, preferably on the axis of symmetry of the applanator, this observation is made even easier.

In order to be able to optimally adjust the size of the contact face of the applanator placed on the eye and/or in order to obtain as exact a cutting area of the pocket as possible, it is recommended that the transparent applanator has markings on its side directed towards the eye, for determining the size of the contact face of the applanator as well as the cutting face on the eye.

If the option of exchanging the applanators for cutting out a lens-shaped portion of tissue is to be dispensed with, this can be achieved if at least the contact face of the applanator is made of a deformable material, which can then be curved by means of an actuator to obtain different shapes that are maintained. The actuator can thus be used to give the applanator inserted in the receptacle differently curved contact faces, so as to ensure that a lens-shaped portion of tissue is cut out.

If the blade holder has an actuator operating perpendicularly with respect to the cutting plane of the blade, it is easily possible for a surgeon to adjust the depth of the pocket cut in the corneal surface, since this actuator can be used to adjust the distance of the blade from the applanator and/or from the contact face of the applanator on the eye.

The blade holder consists, for example, of a lever system comprising at least two lever arms with pivot axes perpendicular to the cutting plane of the blade. If one arm receives the blade and the other arm is articulated on the frame, this provides a simple construction ensuring that the blade can be moved radially with respect to the ring body and also pivoted about an axis perpendicular to its guide plane.

A further option is for the blade holder to comprise a fork-like blade guide, which is guided, as far as possible without clearance, between parallel surfaces of a peripheral groove provided on the frame, in particular on the receptacle. Not only is this embodiment particularly simple in its construction, it also predefines the space within which the surgeon can move the blade.

In order to permit particularly simple insertion and/or exchange of the applanators with differently curved contact faces, it is proposed that the applanator be held in the receptacle in a manner limited by a stop and by using a partial vacuum. The only requirement for this is to provide the receptacle with a pressure line which sucks out air present between the recess and the applanator.

According to another embodiment, a vibrator can be designed and arranged in such a way that the blade executes an oscillating movement with an amplitude of less than 0.2 mm, preferably of less than 0.1 mm, in particular of less than 0.05 mm.

In addition, a vibrator can be designed and arranged in such a way that the blade executes an oscillating movement with a frequency greater than 400 Hertz, preferably greater than 700 Hertz.

The oscillating movement of the blade by a vibrator (vibration) does not represent any relevant direct cutting movement for producing the corneal pocket. The influence of the vibration on the cutting procedure is instead indirect, because of the comparatively small movement amplitude, and is such that the actual blade movement for forming the corneal pocket is made easier by the vibration of the blade and is improved in terms of its precision.

When using a device according to the invention, provision is made that, when the pocket is being cut, part of the blade receptacle penetrates into the cornea.

### BRIEF DESCRIPTION OF THE FIGURES

Illustrative embodiments of the invention are depicted schematically in the drawings, in which:

FIG. 1 shows a partially sectioned side view of a device according to the prior art,

FIG. 2 shows a partially sectioned side view of a second illustrative embodiment of a device according to the prior art,

FIGS. 3a to 3c show plan views of the cuts made by the blade of the devices according to FIG. 1 or FIG. 2,

FIG. 3d shows a plan view of the pocket produced by the cuts made in FIGS. 3a to 3c,

FIG. 4a shows a cross section through a cornea deformed by an applanator, with a blade applied for producing a pocket,

FIG. 4b shows a cross section illustrating the pocket produced according to FIG. 4a, with the cornea undeformed,

FIG. 5 shows the blade secured on the holder, which comprises a fork-like blade guide according to the prior art,

FIGS. 6a to 6c are cross sections showing the cuts made to cut out a lens-shaped portion of tissue using applanators with differently curved contact faces,

FIG. 7 shows a design variant of an applanator with a curvable contact face on an enlarged scale,

FIGS. 8a to 8g show side views of different applanators for the device according to the invention,

FIG. 9 shows a plan view and a side view of a blade according to the invention, and

FIG. 10 shows a plan view of the device into which a transparent applanator has been inserted,

FIG. 11 shows a bottom view (plan view from below) of a blade according to the invention with shaft and cutting area, and also with a roughened area,

FIG. 12 shows a side view of the blade according to the invention adhesively bonded in a blade receptacle, with the cutting edge at the top and the blade receptacle sectioned,

FIG. 13 shows a side view of the blade according to the invention adhesively bonded in a blade receptacle, with the cutting edge in the middle, the blade receptacle sectioned, and with the contact face of the applanator,

FIG. 14 shows a top view of the blade from FIG. 13 adhesively bonded in a blade receptacle, with the cutting edge additionally continued onto the blade receptacle,

FIG. 15 shows the blade from FIG. 13 and FIG. 14 as seen from the pointed tip,

FIG. 16 shows a section through a two-part receptacle,

FIG. 17 shows an illustrative embodiment for limiting the blade movement to what is substantially only a linear forward drive,

FIG. 18 shows an alternative device from the prior art,

FIG. 19 shows an exploded view of the device from FIG. 18,

FIG. 20 shows a plan view of an illustrative embodiment of a blade receptacle according to the invention for a device from FIG. 19,

FIG. 21 shows a partially sectioned side view of an illustrative embodiment of a blade receptacle according to the invention for a device from FIG. 19,

FIG. 22 shows a plan view and side view of an embodiment in which the cutting edge of the blade is continued onto a second blade, which is mounted behind the first blade on the blade receptacle.

### WAY OF IMPLEMENTING THE INVENTION

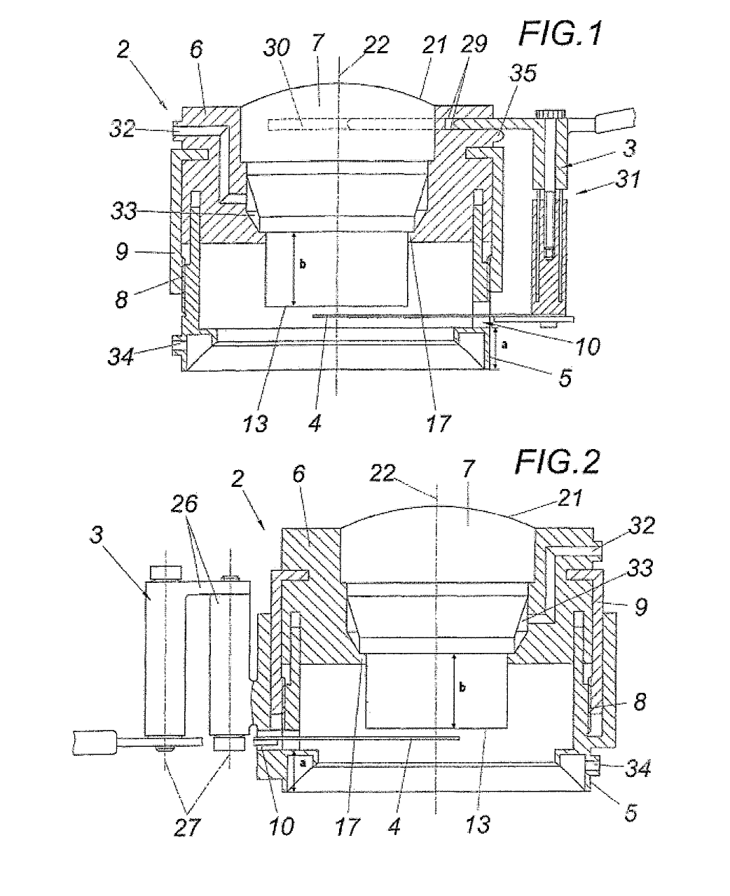

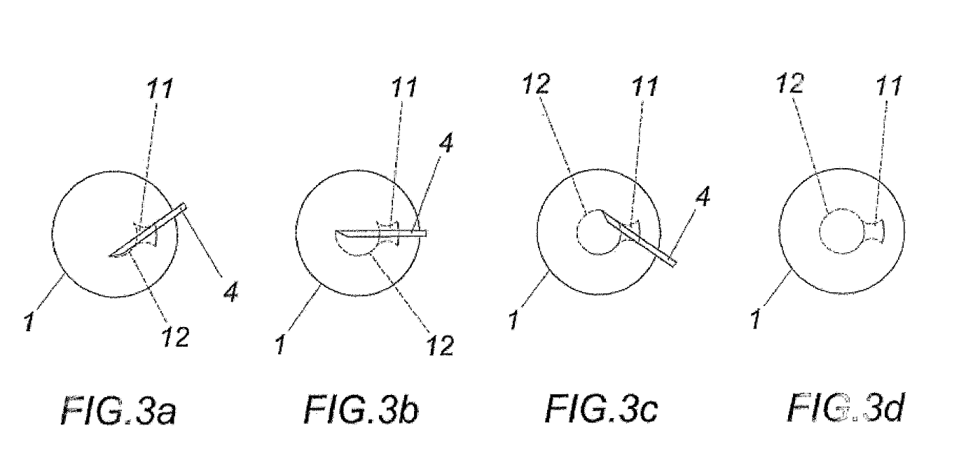

According to the illustrative embodiment in FIG. 1 and the illustrative embodiment in FIG. 2, the devices according to the prior art for cutting a cornea 1 of an eye in order to correct the refractive power thereof generally comprise a frame 2 and a holder 3 for a blade 4. The frame 2 has a ring body 5, which can be suctioned onto the eye, and an applanator 7, which is adjustable coaxially with respect to the ring body 5, and/or a receptacle 6, which is adjustable coaxially with respect to the ring body 5 and serves to receive an applanator 7 for deforming the cornea within the ring body 5. The cornea 1 thus extends through the ring body 5 within which, in particular offset vertically in relation to the ring body 5, the applanator 7 for deforming the cornea is located. For the coaxial adjustment of the receptacle 6, the ring body 5 is provided with a thread 8, in which a nut 9 engages that is mounted rotatably on the receptacle 6. By rotating the nut 9, the receptacle 6 and/or the applanator 7 can thus be adjusted relative to the ring body 5 and/or the cornea 1. If the adjustability of the receptacle 6 is omitted, the same variability can be produced equally through the use of different frame dimensions and/or also applanator dimensions. In each embodiment, the applanator 7 can be moved coaxially with respect to the receptacle 6 and/or pivoted in equally from the side. The holder 3 for the blade 4 is guided on the frame 2 in a plane perpendicular to the axis of the ring body 5, and the blade 4 passes with clearance through a peripheral recess 10 in the frame 2 and is mounted in front of the applanator 7. In particular, the blade 4 is guided by the holder 3 in such a way that, in order to cut into the corneal tissue a pocket 12 having merely a tunnel-like access 11, the blade 4 is on the one hand radially movable with respect to the ring body 5 via the holder 3 and on the other hand pivotable about an axis perpendicular to the guide plane, as can be seen in particular from FIGS. 3a to 3c. It is conceivable that the cutting plane E of the blade 4 also lies in the guide plane of the blade 4.

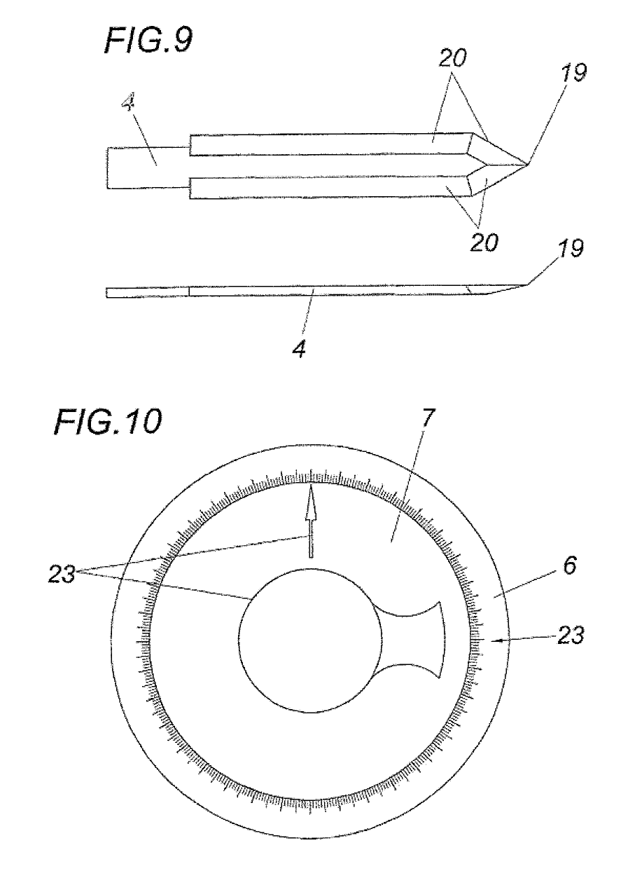

According to the invention, the blade 4 in FIGS. 1 and 2 is replaced by a blade 4 secured on a blade receptacle 50, wherein the blade receptacle 50 is connected to the holder 3. This can be achieved, for example, by means of a fork-like blade guide 28, which is shown in FIG. 5. The blade receptacle 50 in FIG. 5, however, protrudes with its top side above the top side of the blade 4, as can be seen from the fact that, in the blade receptacle 50, the shaft of the blade 4 can be discerned only on account of the cross-sectional view, which shaft, according to FIG. 9, has the same thickness as the blade 4. According to the invention, therefore, the blade receptacle in FIG. 5 is to be reduced in thickness such that its top side does not protrude above the top side of the blade 4.

Accordingly, it is now no longer the blade 4 that passes with clearance through the peripheral recess 10, but at least part of the blade receptacle 50.

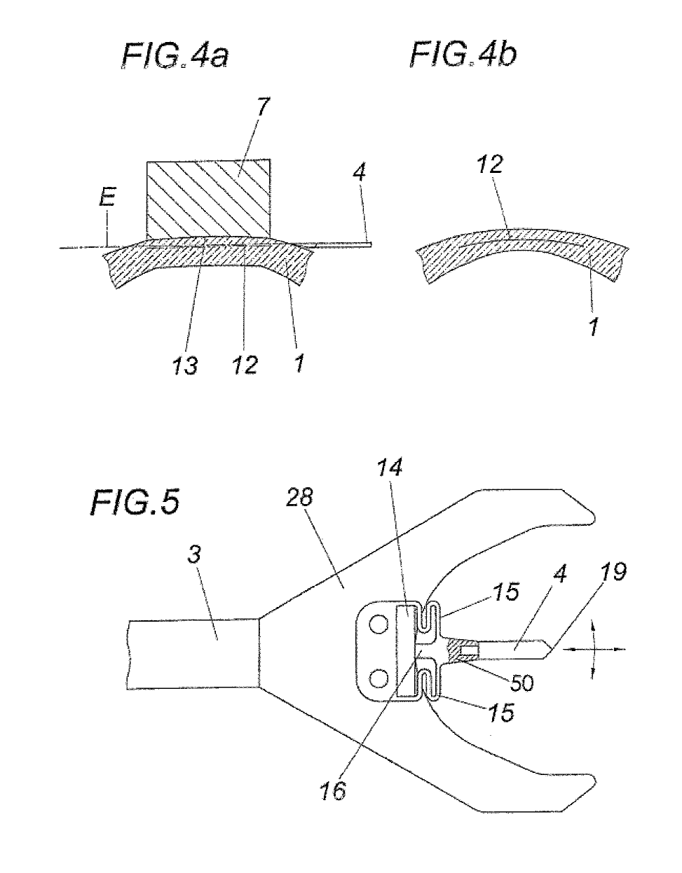

If a pocket 12 is now to be cut into the cornea 1 (FIG. 4a), an applanator 7 is first pressed onto the corneal surface in such a way that the cornea 1 is deformed in a defined manner corresponding to the contact face 13 of the applanator 7, with the shape of the applanator 7 being stamped on the cornea 1. With such pressing of the cornea 1, a suitably large pocket 12 can then be cut into the cornea 1. Once the applanator 7 has accordingly deformed the cornea 1, the blade 4 is applied to the cornea 1 and cuts through the outer tissue layers of the cornea 1 in order to produce a tunnel-like access 11. It is of crucial importance in this respect that the blade 4 does not slip from where it is applied to the corneal surface. The invention therefore comprises on the one hand a blade 4 that passes with clearance through the recess 10 in the frame, and on the other hand the holder 3 supports a vibrator 14 for setting the blade 4 in vibration in the cutting plane E, as can be seen from FIG. 5. By passing through the peripheral recess 10 of the frame free of clearance, the blade 4 does not lie as in the prior art, such that damage to the blade 4, particularly to the edge of the blade 4, can be ruled out. Therefore, particularly hard and brittle materials can also be used for the blade 4, and the resulting sharpness of the blade 4 means that slipping of the latter as it penetrates into the cornea 1 is virtually ruled out. However, it has been found that, despite such blades 4, great force is required to penetrate the outer layer of the cornea 1, and this is made easier, according to the invention, by vibration of the blade 4. For this purpose, the holder 3 preferably has a vibrator 14, which is designed as a piezo element, vibrates in the cutting plane E of the blade 4 and thus acts on the blade 4, which is attached to the holder 3 by springs 15, with the aid of a web 16 bearing on the piezo element. Instead of the piezo element, however, it is conceivable to use an unbalance motor or another suitable source of vibration. Once the tunnel-like access 11 has been cut through the outer tissue layers of the cornea 1, the blade is then guided for the purpose of cutting a pocket 12 in such a way that no further contact with the corneal surface is made. Therefore, a pocket 12 is cut in the cornea 1 only via the tunnel-like access 11, and this cutting is of course also made easier by the vibrator 14 and can also be done with great precision. The pocket 12 and the tunnel-like access 11 can be widened by spreading them using a suitable instrument, by which means the insertion of implants can be made easier. With the aid of another device, a preferably foldable or deformable implant can be introduced into the implant bed in the cornea 1 through the tunnel-like opening 11 that has been formed. The implant then unfolds into the desired shape in the pocket 12.

Since the invention allows a very exact pocket 12 to be created inside a cornea 1, it is possible, among other things, to implant lenses with a standardized radius of curvature into the area of the optical centre. This is significant because the natural curvature of the cornea varies between about 7 mm and 9 mm and, with a flat, uncurved impingement of the cornea 1, a defined radius of curvature of the pocket 12 may not be achieved. By using an applanator 7 with a defined curvature of the contact face 13 and by fixing a defined cutting depth, the radius of curvature of the pocket 12 to be produced can be exactly defined, and this means that lenses that are standardized in terms of the curvature of their surface can be implanted in all patients, thus providing considerable cost savings in lens production.

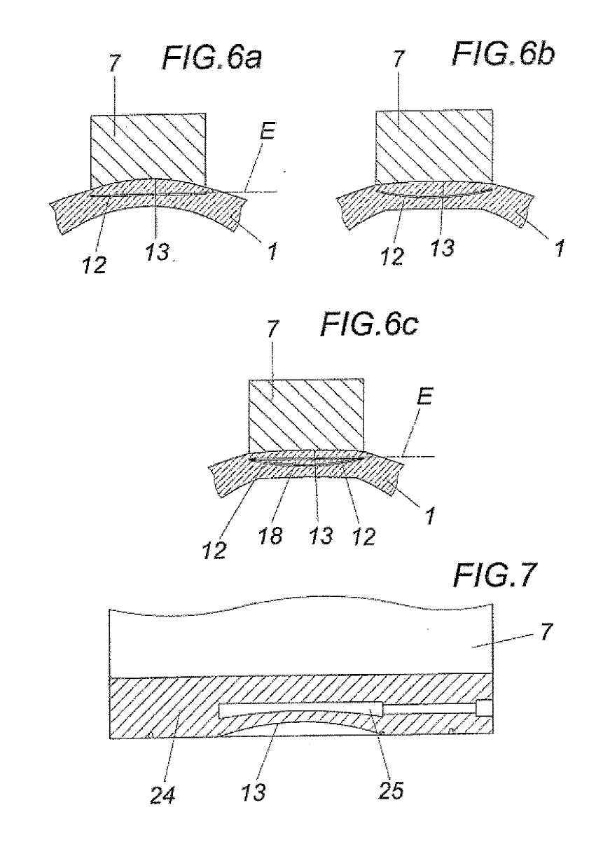

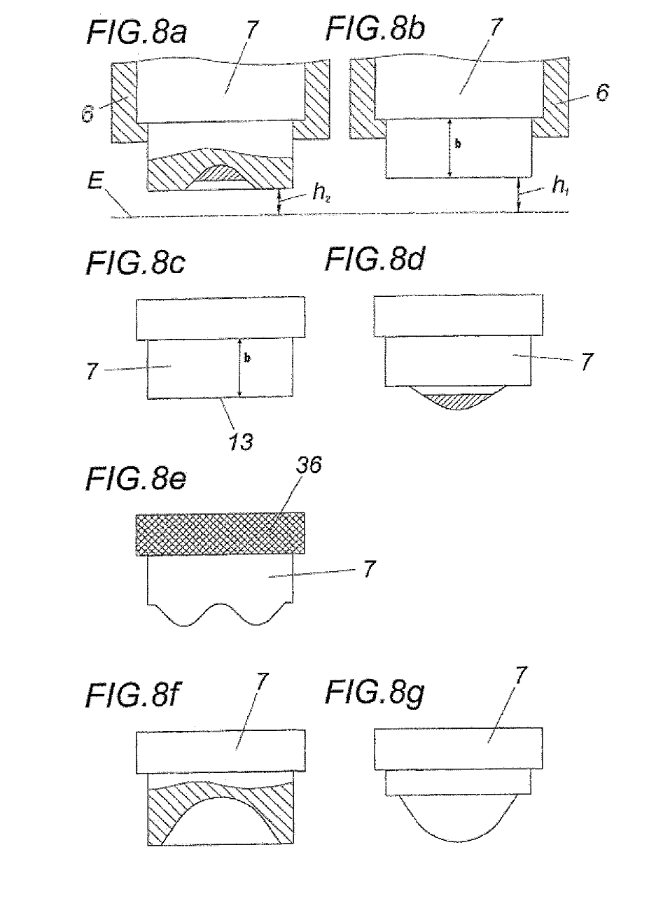

The receptacle 6 for the applanator 7 is designed as an exchangeable receptacle, in which applanators 7 with differently curved contact faces 13 for deforming the cornea can be received in a manner limited by a stop. This stop 17 can be seen in particular in FIGS. 1 and 2. In this way, pockets 12 for delimiting a lens-shaped portion of tissue 18 can be easily produced by performing successive incisions. In particular however, since the frame 2 does not need to be removed from the eye when changing applanators, an exchangeable receptacle ensures that a lens-shaped portion of tissue 18 with a predefined size is always cut out, which is not the case in the known devices. The lens-shaped portion of tissue 18 can be produced according to FIGS. 6a to 6c. After a first pocket 12 has been cut into the corneal tissue, as has also been described above, the blade 4 is removed from the corneal tissue (FIG. 6a) and a second pocket 12, stamped by another applanator 7 with a differently curved contact face 13, is cut (FIGS. 6b and 6c). For this purpose, the applanator 7 either has to be changed or have its contact face 13 altered, as is possible, for example, using an applanator 7 according to FIG. 7. A further incision through the tunnel-like access 11 not only produces a new pocket 12 but also cuts out a lens-shaped portion of tissue 18 (FIG. 6c) which, for example, can be removed through the tunnel-like access 11 using tweezers. The lens-shaped portion of tissue corresponds in form to the difference of the differently curved contact faces 13 of the two applanators 7 and/or the desired dioptric change through corresponding alteration of the anterior corneal curvature. The applanator 7 also has, in its upper area, a grip 36 with an engraved surface (FIG. 8e), which makes the applanator 7 easy to change. It is useful in this case if the applanator 7 protrudes from the receptacle 6 so as to be easily grasped.

To make it easier to cut out a lens-shaped portion of tissue 18, the blade 4 for the second incision can be inserted somewhat deeper into the cornea 1 (FIGS. 8a and 8b). This can be achieved by ensuring that the perpendicular distances h1 and h2 of the peripheral edges of the contact faces 13 of the applanators 7, successively inserted into the receptacle 6, are different with respect to the cutting plane E of the blade 4. Illustrative embodiments of different contact faces 13 of applanators 7 are shown in FIGS. 8a to 8g. The dimensions of the lens-shaped portion of tissue 18 that is cut out using the applanators according to FIGS. 8a and 8b and FIGS. 8c and 8d are indicated by hatching.

According to FIG. 9, the preferably non-metallic blade forms a pointed tip 19 with two blade edges 20 originating from this tip. This specific type of pointed tip 19 has proven useful in particular for penetrating the outer layer of the cornea 1 and/or for producing a tunnel-like access 11. The shape of the blade 4 resembles that of a double-edged knife.

The applanators 7 are preferably made of transparent material, such as plastic or glass, and are designed, as in FIGS. 1 and 2, as magnifying lenses 21, with their focal point lying in the area of the contact face 13, preferably on the axis of symmetry 22 of the applanator. With an applanator 7 of such a design, it is quite easy for a surgeon to monitor the progress of treatment of the cornea 1.

The transparent applanator 7 has markings 23 on the side directed towards the eye. These markings 23 allow a surgeon to optimally orientate himself, for example in terms of where to apply the blade 4 for cutting a tunnel-like access. Markings 23 can also be applied for the optical treatment zone in order to indicate to the surgeon the boundaries of the pocket 12 that is to be cut. The transparent applanator 7 also has markings 23 on its side directed away from the eye, which are related to markings 23 on the receptacle 6 for the applanator 7 (FIG. 10). This allows the surgeon, by rotating the applanator 7 in relation to the receptacle 6, to perform refractive power corrections, especially in cases of astigmatism.

The contact face 13 of the applanator shown in FIG. 7 is made of a deformable material 24, which can be curved via an actuator 25 in order to assume different shapes that are maintained. Through a supply line to the actuator 25, compressed gas and compressed liquid or the like can be fed into the cavity of the actuator 25, by which means the contact face 13 of the applanator 7 can be curved so as to obtain different shapes that are maintained.

According to the illustrative embodiment in FIG. 2, the holder 3 consists of a lever system comprising at least two lever arms 26 having pivot axes 27 that are perpendicular to the cutting plane E of the blade 4, wherein one lever arm 26 receives the blade 4, and the other lever arm 26 is articulated on the frame 2, preferably on the receptacle 6.

According to the illustrative embodiment in FIG. 1, the holder 3 can also comprise a fork-like blade guide 28, which receives the blade 4 and which is guided, as far as possible free of clearance, between parallel surfaces 29 of a peripheral groove 30 provided on the frame 2, in particular on the receptacle 6. The blade 4 is offset in relation to the fork-like blade guide 28, and the distance between the cutting plane E of the blade 4 and the contact face 13 of the applanator 7 is adjustable by means of an actuator 31 in the form of a screw drive. A knob 35 is provided on the receptacle 6 in order to ensure that the fork-like blade guide 28 can be easily pushed in.

The applanator 7 can be fixed in the receptacle 6 by means of a partial vacuum. For this purpose, air can be sucked out of a chamber located between receptacle and applanator by way of a line 32. For this purpose, the applanator has the shape of a truncated cone, which allows easy insertion of the applanator. It is also conceivable, instead of the pressure line, to use other mechanical holders, for example a bayonet closure, or magnetic, electromagnetic, hydraulic or equivalent mechanisms. The ring body 5 is suctioned onto the eye in a similar way via a pressure line 34.

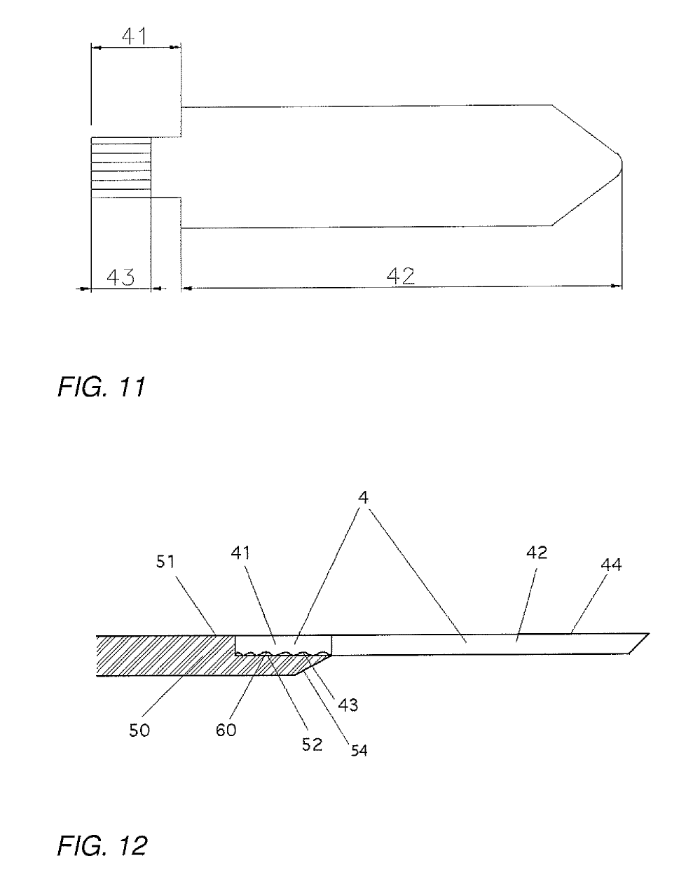

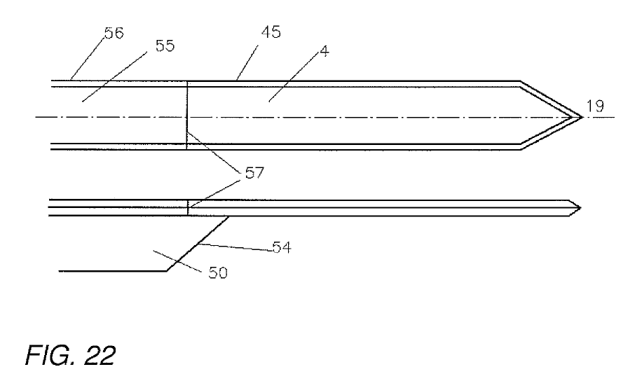

It will be seen from FIG. 11 that the blade 4 has a shaft 41, for securing the blade 4 in the blade receptacle 50, and a cutting area 42, and also a roughened area 43 on at least one side of the shaft 41, which can be a partial area of the surface of the underside of the shaft 41. The cutting area 42 is the longitudinal area of the blade which has a blade edge and with which cuts can therefore be made. The longitudinal area of the blade that does not have a blade edge, for example the shaft 41 of the blade, does not belong to the cutting area. The shaft can be narrowed in relation to the cutting area 42 (FIG. 11) or of the same width (FIG. 22) or, in special cases, it can even be wider, for example if the cutting edge is continued onto the blade receptacle or onto a second blade.

According to FIG. 12, the blade 4 is now adhesively bonded into the blade receptacle 50 in such a way that the flat top side 51 of the blade receptacle 50 (the side directed towards the applanator or contact face 13) does not protrude above the flat top side 44 of the blade. In this example, the flat top side 51 of the blade receptacle 50 and the flat top side 44 of the blade form a common flat surface. The cutting edge is indicated here on the flat top side 44 of the blade, but it can also extend centrally between the top side 44 and underside 46 of the cutting area 42 of the blade 4. Ideally, not only is the underside of the shaft 41 roughened, or a partial surface 43 thereof, but also a corresponding surface of the blade receptacle 50, which is designed here as a depression 52 in the area where the shaft 41 is received in the blade receptacle 50. The adjoining surfaces 43 of the blade and 52 of the blade receptacle are connected by means of a suitable adhesive 60.

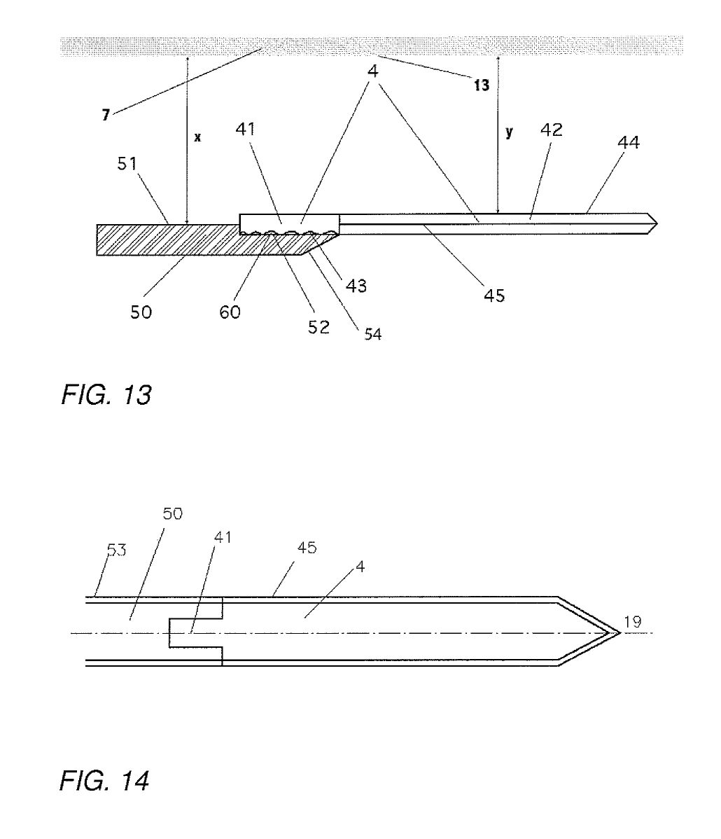

In another embodiment, according to FIG. 13, the top side 51 of the blade receptacle is not intended to protrude above the cutting edge 45 of the blade 4, for which reason a corresponding depression 52 is provided. Here, in contrast to FIG. 12, the cutting edge 45 is arranged in the centre between the top side 44 and underside 46 of the blade. The roughened area 43 and the roughened depression 52 are again connected to each other by means of adhesive 60. A poorer design variant is one in which, with a symmetrical cutting edge (in the centre between the top side and underside of the blade), the top side 51 of the blade receptacle 50 protrudes above the cutting edge 45 of the blade 4 but does not protrude above the top side 44 of the blade 4.

The applanator 7 with the contact face 13 is also indicated in FIG. 13. It will be noted that, during the cutting procedure, the distance x between the highest point of the flat surface on the top side 51 of the blade receptacle 50 and the contact face 13 of the applanator 7, measured perpendicular to the cutting plane, is not less than the distance y between the flat surface on the top side 44 of the blade 4 and the contact face 13 of the applanator 7, measured perpendicular to the cutting plane E.

FIG. 14 shows a plan view of the blade from FIG. 13. The blade 4 is designed as a longitudinal blade with two cutting edges 45, which extend along the longitudinal sides and which taper towards a pointed tip 19 at the end directed away from the blade receptacle. The cutting edges 45 extend in parallel along both longitudinal sides of the blade and taper towards the pointed tip 19 at an angle alpha of ca. 70° at the end directed away from the blade receptacle.

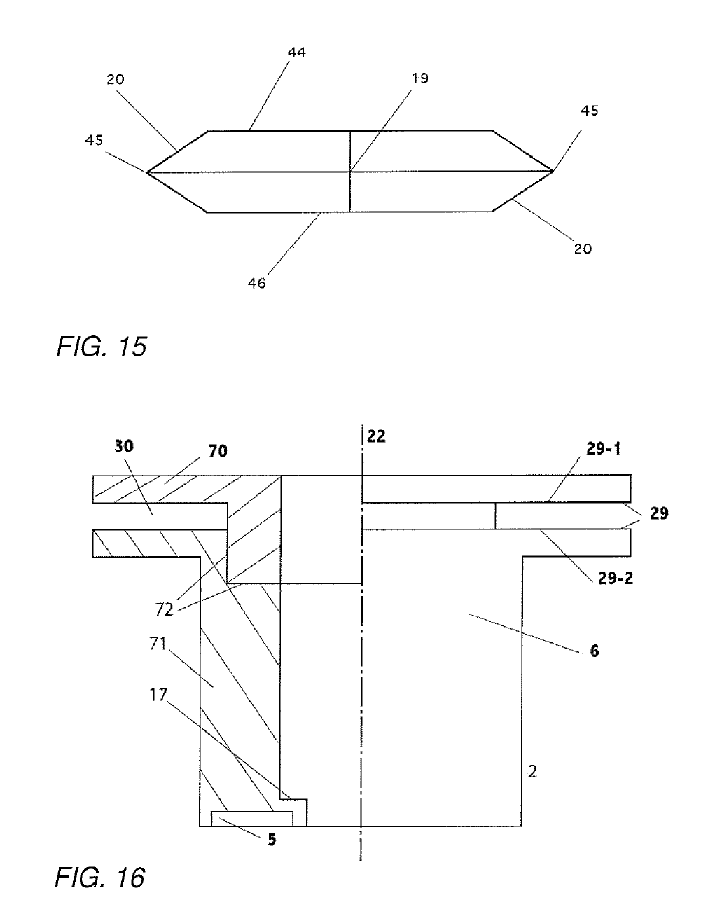

The blade edge 20, i.e. the areas bevelled in relation to the flat top side and underside of the blade and forming the cutting edge 45 at their line of intersection, is bevelled with respect to the surface by an angle beta of about 15° (see FIG. 15).

From the shaft to the blade tip (=pointed tip, the end directed away from the blade receptacle), the cutting edge has a length of at least 3 mm and preferably 5 mm. The cutting edge 45 or blade edge 20 is continued with an identical profile into the blade receptacle 50 (see reference sign 53). The continuation 53 of the cutting edge onto the blade receptacle can be up to 7 mm long, ideally 5 mm, such that the blade can effectively produce a cut of up to 12 mm (ideally of between 5 mm and 12 mm).

The blade is preferably designed with a thickness of below 300 micrometres, ideally between 100 micrometres and 200 micrometres, such that the cutting edge is preferably constantly 50 to 100 micrometres equidistant from the blade surfaces 44, 46. The blade is ca. 2 mm wide and, at the end directed towards the blade receptacle 50, has a shaft 41, which is ca. 2 mm long and at least 1 mm wide. The geometry of the shaft can in principle be of any kind. The thickness of the shaft in this illustrative embodiment is identical to the thickness of the blade. On the side directed away from the applanation face 13, the shaft 41 is roughened (reference sign 43) compared to the rest of the blade surface. The rest of the blade surface is preferably lapped and very smooth. The roughened surface should measure at least 1 mm², but ideally 2 mm² or more. The three side surfaces of the shaft can also be roughened.

The shaft can of course also be thicker or thinner than the cutting blade part.

The freedom of movement of the blade 4 in the cutting plane (cutting surface) E is at least in the radial direction in each position within the cutting surface. The blade is radially movable in different directions and pivotable about an axis perpendicular to the cutting plane. The blade in each position within the cutting surface can be oriented and moved freely in all directions of the cutting plane. Moreover, a limiting mechanism can be provided, for example for limiting the amplitude of a reciprocating movement to defined levels of the amplitude along the forward movement of the blade. In the preferred design variant, such a limitation of the amplitude of a lateral movement is not provided. In each case, an additional vibration, which does not cause any actual cutting when the blade is at rest (no radial and/or pivoting movement), can be included as third movement component. Thus, a maximum of three different blade movements can be superposed in each point of the physical cutting plane (radial movement, pivoting movement, vibration). The vibration is preferably of such small excursion and of sufficiently high frequency that it cannot be seen by the naked eye. The amplitude is preferably less than 0.2 mm (ideally less than 0.1 mm or even less than 0.05 mm) and the frequency preferably greater than 400 Hertz (ideally greater than 700 Hertz).

The applanator 7 should have a height (along the centre axis) of more than 1 cm. The peripheral recess 10 of the frame 2, for the passage of the blade 4 through the frame 2, should be not more than 5 mm away from the lower edge of the ring body 5 (ideally between 2 and 5 mm) (see reference sign a in FIG. 1 and FIG. 2). The stop provided for the applanator 7 in the receptacle 6 should be not more than 4 mm (ideally between 0.5 mm and 2 mm) from the applanation face (=contact face) 13 of the applanator 7 fixed in the receptacle 6 (see reference sign b in FIG. 1, FIG. 2 and FIG. 8.

The applanator 7 can also be fixed in the frame 2 with a lever device for example.

The blade receptacle 50 can be an integral part of the holder 3 for the blade 4 guided on the frame 2, i.e. can be formed in one piece with the holder 3. In the illustrative embodiment according to FIG. 5, the blade receptacle 50 would accordingly be part of the fork-like blade guide 28.

The receptacle 6 from FIGS. 1 and 2 can also be designed in two parts in accordance with FIG. 16, such that an upper receptacle part 70 with a flange-like continuation forms an upper surface 29-1 of the peripheral groove 30, while a lower receptacle part 71 with a flange-like continuation forms a lower surface 29-2 of the peripheral groove 30. The two receptacle parts 70, 71 are connected to each other by a connection 72, preferably an adhesive, or by other means, such as a form fit or a thread. The upper receptacle part 70 lies in a recess on the lower receptacle part 71.

By virtue of the fact that one surface 29-1 belongs to one part 70 of the receptacle and the other surface 29-2 belongs to the other part of the receptacle and the two are then suitably connected to each other, it is possible, for example by means of a turning machine, to produce both surfaces exactly perpendicular to the axis 22. If this is not the case, in other words if the peripheral groove 30 is made in a one-part receptacle, there is a danger, particularly in the case of very hard material, of the two surfaces 29-1, 29-2 not extending exactly in parallel. If the receptacle is designed in two parts, the surfaces 29-1 and 29-2 can be turned flat from “in front”, whereas in a one-part design the peripheral groove 30 has to be turned using a “grooving chisel”. It has been found that the latter method gives much poorer parallelism of the surfaces 29-1, 29-2 than the first method. In particular, in the latter method, the peripheral groove 30 will be somewhat thicker in the outer area away from the axis 22, in other words the surfaces 29-1, 29-2 lie further apart there than they do centrally (near the axis). This can lead to considerable imprecision of cutting, with a risk to the patient's eyesight.

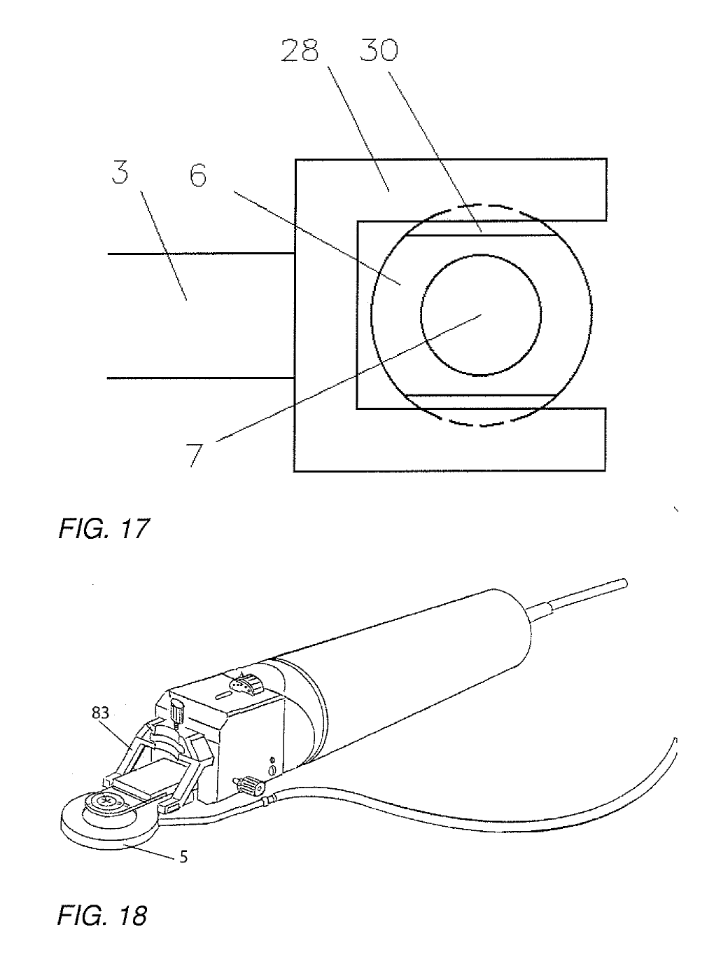

FIG. 17 shows an illustrative embodiment for limiting the blade movement to what is substantially only a linear forward drive. This is obtained if the device according to the invention has no vibrator and/or if the fork-like blade guide 28 from FIG. 5 and the peripheral groove 30 on the receptacle 6 of the frame 2 for the applanator 7 are designed such that only a linear or radial movement (forward drive) of the blade is possible in the cornea, that is to say without substantial pivotability or reciprocating movement. A restriction to a merely radial or linear movement can be achieved, for example, if the outer sides of the peripheral groove 30 and the inner sides of the fork-like blade guide 28 extend exactly parallel to each other and without clearance or in practice with only minimal clearance.

In another design variant, but one which is preferably suitable for producing comparatively small corneal pockets and has no frame 2 through which the blade can be passed with clearance, for example according to U.S. Pat. No. 6,599,305 B1, the safety of the patient can be greatly improved by a blade receptacle and blade according to the invention and by securing of the blade (adhesion and, if appropriate, roughening).

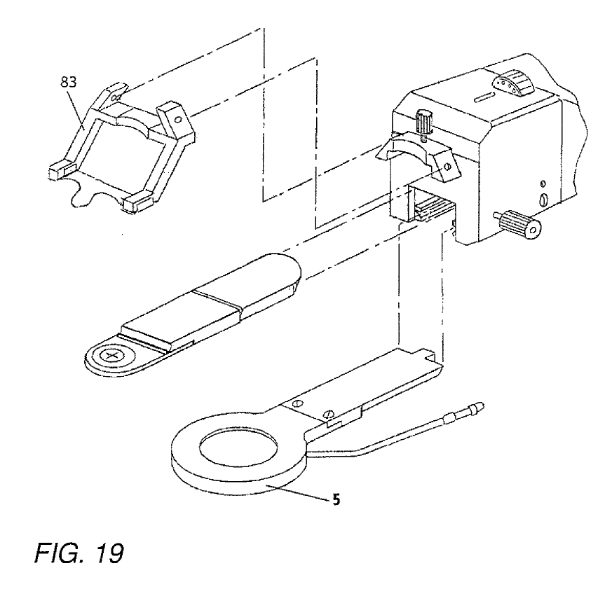

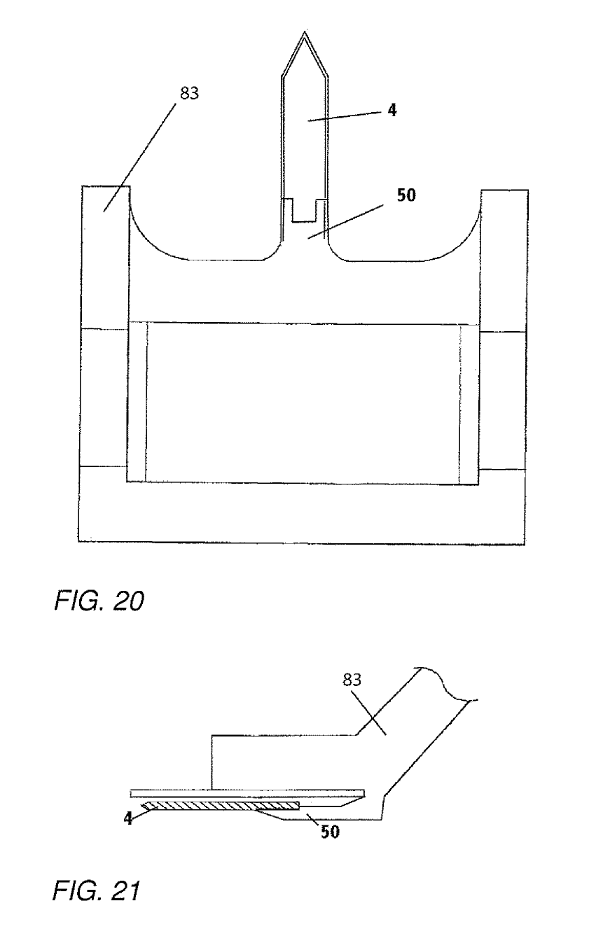

FIG. 18 and FIG. 19 show a corresponding design from the prior art according to U.S. Pat. No. 6,599,305 B1. They show a device that also has a ring body. 5. A fixture 83, which has both a blade and also an applanator, the latter at a fixed distance from the blade, can be pushed forward by the device. The blade oscillates, under the action of the fixture 83, about the forward direction with an amplitude that determines the width of the pocket. The blade is clamped on both sides (laterally) into the fixture 83. Therefore, the only material suitable in practice for such a blade is a ductile material (metal), since solid materials such as diamond, ceramic, etc., would break during lateral clamping in the fixture 83. Moreover, such a large diamond, for example, would be difficult to produce and extremely expensive, among other reasons because of only limited availability on the market. Blades made of metal, however, can in practice be used only as disposable blades and are extremely unstable (bendable) in this size, which increases the tolerance of the fit of each blade and thus also increases the imprecision when cutting. However, if the fixture 83 and the blade were designed in one piece, then hard materials such as ceramic could also be used.

If, according to the invention, a blade receptacle is used which is secured on the fixture 83, then materials can also be combined. For example, a blade receptacle made of metal, semiconductor, plastic or ceramic with a blade made of any desired material, but preferably of any desired hard material such as diamond, ruby, ceramic, etc. Such a design variant is seen in FIG. 20 and FIG. 21 for example. There, a blade receptacle 50, which supports the blade 4, is clamped in the fixture 83. According to the invention, the top side of the blade receptacle 50 does not protrude above the top side of the blade 4 or the cutting edge thereof. The blade 4 is shown in section in FIG. 21. In terms of its function of securing the blade receptacle, the guide 83 is equivalent to the holder 3.

In a particularly advantageous embodiment of the invention, the cutting edge of the blade is continued onto the blade receptacle by up to 7 mm, ideally by 5 mm, such that a blade can effectively produce a cut of up to 12 mm (ideally between 5 mm and 12 mm) (see FIG. 14). The cutting edge 45 of the blade 4 is continued in a plane extending on the blade receptacle 50 merging into each other. In a particular design variant according to FIG. 22, this cutting edge 45 can also be formed on the blade receptacle 50 by a second blade 55 arranged there. The cutting edge 45 of the blade 4 then continues into the cutting edge 56 of the blade 55. Typically, the length of the cutting edge 45 is between 4 and 6 mm (ideally 5 mm) and that of the cutting edge 56 is between 2 and 6 mm (ideally 4 mm). The boundary line 57 between both blades 4, 55, which then abut each other, can be of any desired form and is straight in FIG. 22. The blade 4 in FIG. 22 has no shaft 41 in the real sense and consists merely of a cutting area 42. In principle, the second blade 55 could also be omitted, in which case the blade 4 would then be maintained in its form without an actual shaft. If appropriate, however, that part of the blade 4 that lies directly over the blade receptacle 50 or overlaps the latter can be designated as shaft 41.

The shaft geometry can in principle be of any kind.

Generally, an oscillation or a vibration can be provided to support the cutting movement. It is of course also possible to work without oscillation and vibration.

An oscillating cutting movement is to be understood here as meaning that, when the blade 4 is periodically moved perpendicular to the forward drive axis, this excursion about the forward drive axis represents a relevant cutting movement, and the incision into the tissue along the excursion thus determines and/or at least directly contributes to the pocket dimension and pocket size in the cornea. By contrast, the movement of the blade by a vibrator (vibration) does not represent any relevant direct cutting movement for producing the corneal pocket. The pocket dimension or pocket size is not determined or not essentially determined by the direct blade movement resulting from the vibration. The influence of the vibration on the cutting procedure is instead indirect, because of the comparatively small movement amplitude, and is such that the actual blade movement for forming the corneal pocket is made easier by the vibration of the blade and is improved in terms of its precision.

In principle, the various elements of the embodiments can be combined to form new embodiments.

### LIST OF REFERENCE SIGNS

1 cornea
2 frame
3 holder
4 blade
5 ring body
6 receptacle for an applanator 7
7 applanator
10 peripheral recess of the frame 2
11 tunnel-like access of the pocket 12
12 pocket in the corneal tissue
13 contact face (applanation surface or applanator)
14 vibrator
17 stop in the receptacle 6 for the applanator 7
18 lens-shaped portion of tissue
19 pointed tip of the blade 4
20 blade edge
21 magnifying lens of the applanator 7
22 axis of symmetry of the applanator
23 markings on the applanator 7
24 deformable material of the contact face 13 of the applanator 7
25 actuator for deformable material 24
26 lever arm of the holder 3
27 pivot axes of the lever arms 26
28 fork-like blade guide
29-1 surface of the peripheral groove 30 on the upper holder part 70
29-2 surface of the peripheral groove 30 on the lower holder part 71
30 peripheral groove on the frame 2
31 actuator
32 line
35 knob on the receptacle 6
41 shaft of the blade 4
42 cutting area
43 roughened area of the shaft 41 of the blade 4
44 top side of the blade 4
45 cutting edge of the blade 4
46 underside of the blade
50 blade receptacle
51 top side of the blade receptacle 50
52 roughened depression of the blade receptacle 50
53 cutting edge on the blade receptacle 50
54 underside of the blade receptacle
55 second blade on the blade receptacle 50
56 cutting edge of the second blade 55
57 boundary line between blade 4 and blade 55
60 adhesive
70 upper receptacle part
71 lower receptacle part
72 connection between receptacle parts 70, 71
83 fixture for receiving the blade

1. Device for cutting the cornea (1) of an eye in order to correct the refractive power thereof, with a ring body (5) that can be suctioned onto the eye, with an applanator (7) for deforming the cornea, and with a blade (4) which is guided in a guide plane perpendicular to the axis of the ring body (5), is mounted in front of the applanator (7) and is used to cut a pocket in the corneal tissue, wherein the blade (4) is secured on a blade receptacle (50) of a holder (3) or fixture (83) in such a way that the top side (51), that is the surface directed towards the contact face of the applanator, of the blade receptacle (50) does not protrude above the top side (44), that is the surface directed towards the contact face of the applanator, of the blade (4), at least within the depth of penetration of the blade receptacle (50) into the cornea (1).

2. Device according to claim 1, wherein the blade (4) is provided with a shaft (41), which is adhesively bonded into the blade receptacle (50).

3. Device according to claim 1, wherein the blade, in particular the shaft (41), is roughened on the surface within a partial area (43), on at least one side, and the roughening is adhesively bonded to the blade receptacle (50).

4. Device according to claim 3, wherein the roughening of the blade (4) and/or of the shaft (41) involves an increase of the average surface roughness, in relation to the rest of the blade surface, by at least a factor of 1.1, preferably a factor of 2 to 100.

5. Device according to claim 3, wherein the blade receptacle (50) is likewise roughened in an area (52) corresponding to the roughened area (43) of the blade (4) and/or of the shaft (41).

6. Device according to claim 1, wherein the blade receptacle (50), on the side directed towards the applanator (7), has a depression (52) corresponding to the dimensions of the blade (4) and/or of the shaft (41).

7. Device according to claim 1, wherein, when the cutting edge (45) of the blade (4) is not located on the top side (44) of the blade (4), the top side (51) of the blade receptacle (50) does not protrude above the cutting edge in the direction of the applanator.

8. Device according to claim 1, wherein the blade (4) is designed as a longitudinal blade with a blade edge (20) along at least one longitudinal side, preferably on both longitudinal sides, and its width is not more than 4 mm, preferably 2 to 2.5 mm.

9. Device according to claim 1, wherein the underside of the blade receptacle (50) extends away from the shaft (41) and from the blade (4) and/or slopes from the lateral edges of the blade receptacle to the center of the blade receptacle.

10. Device according to claim 1, wherein the top side (51) of the blade receptacle (50), at least as far as the blade receptacle penetrates into the cornea, is flat and preferably extends parallel to the cutting plane (E).

11. Device according to claim 1, wherein the blade has a pointed tip (19).

12. Device according to claim 11, wherein the blade (4) has at least one blade edge (20) tapering towards the pointed tip (19).

13. Device according to claim 12, wherein a blade edge (20) is provided on each longitudinal side of the blade (4), and the cutting edges (45) thereof are at the same distance from the top side (44) of the blade as from the underside (46).

14. Device according to claim 13, wherein the two cutting edges (45) extend parallel to each other and, at the end directed away from the blade receptacle (50), taper at an angle of less than 90°, preferably of between 60° and 80°, in particular of approximately 70°, towards the pointed tip (19).

15. Device according to claim 12, wherein the surface of the blade edge (20) is inclined by approximately 10° to 25° towards the top side (44) and/or underside (46) of the blade.

16. Device according to claim 12, wherein the blade receptacle (50), in an area adjoining the blade (4), has the same cross section as the blade, such that the shape of the blade edges (20) and cutting edges (45) of the blade (4) continues by up to a length of 7 mm, in particular by a length of approximately 5 mm, past the blade on the blade receptacle (50), in particular on a second blade (55) integrated in the blade receptacle (50).

17. Device according to claim 1, wherein the blade receptacle (50) is made of metal, ceramic or hard plastic.

18. Device according to claim 1, wherein a vibrator (14) is designed and arranged in such a way that the blade executes an oscillating movement with an amplitude of less than 0.2 mm, preferably of less than 0.1 mm, in particular of less than 0.05 mm.

19. Device according to claim 1, wherein a vibrator (14) is designed and arranged in such a way that the blade executes an oscillating movement with a frequency greater than 400 Hertz, preferably greater than 700 Hertz.

20. Device according to claim 1, wherein a receptacle (6) provided for the applanator (7) is designed in two parts, the two parts together forming a peripheral groove (30) for guiding a holder (3) for the blade (4).

21. Device according to claim 1, with a frame (2) comprising both the ring body (5), which can be suctioned onto the eye, and also a receptacle (6) for an applanator (7) that can be adjusted coaxially to the ring body (5) in order to deform the cornea within the ring body (5), and with the holder (3) that is guided on the frame (2) in a guide plane perpendicular to the axis of the ring body (5) and serves to hold a blade (4), which blade (4) passes through a peripheral recess (10) in the frame (2), is mounted in front of the applanator (7) and, in order to cut in the corneal tissue a pocket (12) having merely a tunnel-like access (11), is on the one hand radially movable relative to the ring body (5) via the holder (3) and on the other hand pivotable about an axis perpendicular to the guide plane at each blade position within an area of a cutting plane parallel to the guide plane, wherein the blade (4) passes through the peripheral recess (10) with clearance, and the holder (3) supports a vibrator (14) for an oscillating movement of the blade (4) in the cutting plane (E).

22. Device according to claim 21, wherein at least part of the blade receptacle (50) passes through the peripheral recess (10) with clearance.

23. Use of a device according to claim 1, wherein, when the pocket is being cut, part of the blade receptacle (50) penetrates into the cornea (1).
